(19) United States
(12) Patent Application Publication
(10) Pub. No.: US 2021/0104334 A1
(43) Pub. Date: Apr. 8, 2021

(54) METHOD FOR PREDICTING A DISEASE OUTBREAK

(71) Applicant: AIME HEALTHCARE SDN BHD, Kuala Lumpur (MY)

(72) Inventors: Mohd helmi Bin ZAKARIAH, KLANG (MY); Dhesi Baha Raja A/L SELVA RAJA, KUALA LUMPUR (MY); Rainier Christopher Domingo MALLOL COTES, Miami, FL (US)

(21) Appl. No.: 17/062,254

(22) Filed: Oct. 2, 2020

(30) Foreign Application Priority Data
Oct. 4, 2019 (MY) PI2019005893

(51) Int. Cl. G16H 50/80; G06K 9/62; G06N 5/00; G06F 17/18

(52) U.S. Cl. CPC G16H 50/80; G06F 17/18; G06N 5/003; G06K 9/6298

(57) ABSTRACT

The present invention relates to a method for predicting a disease outbreak. This method includes the steps of collecting data by the remote data interface, validating the obtained data by the remote data interface, analysing data and computing new parameters by a data analysis interface, identifying the association between the case, the outbreak and various indicators, and establishing one-to-one relationships between each case and the index case of the outbreak, and one-to-many relationships between each case and other cases of the outbreak by the remote data interface, predicting case parameters by a data prediction interface, wherein the predicted case parameters include time of the outbreak and number of people affected by the outbreak, predicting severity of the outbreak by the data prediction interface, and predicting geographical location of an epicentre of the outbreak by the data prediction interface.

### CROSS-REFERENCE TO RELATED APPLICATIONS

The current application claims priority to Malaysia Patent Application No. PI2019005893, entitled “A METHOD FOR PREDICTING A DISEASE OUTBREAK,” filed on Oct. 4, 2019, the contents of which are incorporated by reference in their entireties.

### FIELD OF INVENTION

The present invention relates to a method for predicting a disease outbreak. More particularly, the present invention relates a method to predict parameters of an outbreak of a vector-borne disease.

### BACKGROUND OF THE INVENTION

Diseases such as dengue or Zika impose a very large socioeconomic burden on the region that they are located in. Large amounts of funds have been spent in an attempt to suppress the outbreaks of diseases such as these. In addition, these diseases are potentially lethal and can cause death and severe pain to people infected by them.

Therefore, important decisions have to be made by public health professionals to decide on the steps that need to be taken to minimize the effect of the outbreak. However, due to the fact that there is no direct indicator of the outbreaks, information about the outbreak is usually delayed. This lack of information in the appropriate time causes the decisions to become uninformed. A slight delay in time may also cause the exponential spread of the disease which requires further investment and spending.

Methods such as fumigation and genetically modified mosquitoes have been proven to be effective in managing these diseases, but their potential is limited by the zones in which they are applied. In addition, when professionals do not know where to focus fumigation and genetically modified mosquitoes, and at what point in time, resources are invested in the wrong way. Having prior knowledge of the outbreak before it happens can make combating it easier, by giving the authorities adequate time to prepare the precautions, and preventing the disease from spreading. However, it is very difficult to predict the disease using classical techniques, due to the fact that there is a very large amount of cases to process and a major difficulty in relating cases of a similar disease. The tasks may prove to be impossible for human beings. Hence, there has been development in the field of disease outbreak prediction.

An example of a method for detecting an outbreak of a disease is disclosed in a United States Patent Publication No. 2017/0169182 A1 which relates to a system, computer-readable media, and method for detecting the outbreak of a disease. The method comprises the step of receiving reports of patients with symptoms related to a medical condition in a geographic area, storing the data in a repository, determining whether the reports are associated with a specified component, using an artificially intelligent model to determine the outbreak of the specified condition in a geographic area, and providing an indication of the disease in the geographic area using an output method. However, this method only detects an outbreak after it has already happened, this is also ineffective due to the fact that it may happen in unexpected location and time. If the outbreak happens in unexpected circumstances, there exists a risk of the disease spreading before taking necessary measures to limit it.

Another example of a system and method for predicting disease outbreaks is disclosed in a United States Patent Publication No. 2017/0161617 A1 which relates to a system and method for predicting a disease outbreak using crowdsourced reports of environmental conditions. The method comprises receiving crowdsourced reports containing information about environmental conditions, filtering the reports and extracting relevant parameters. Furthermore, the reports are clustered based on similar contexts, correlated with historical data regarding the disease. The next step involves estimating the probability of the disease outbreak as well as the time in which it will occur and its severity. These parameters are then inputted into an outbreak model to predict the parameters of the outbreak. Finally, the predicted parameters are sent to relevant parties and one or more corrective actions are taken.

This method can predict the outbreak in advance. However, this method involves the use of unreliable sources of information such as social media, mobile applications, search engines, and crowdsourced reports. The unreliable sources of information may lead to errors in the prediction. This is because the information obtained from ordinary people can be ill-informed and not have the required detail to diagnose a specified disease. In addition, the information obtained from social media or search engines can be largely comprised of rumours and false information, the rumours and false information could negatively affect the accuracy of the prediction. Therefore, there is a need for a method to accurately predict disease outbreaks.

### SUMMARY OF INVENTION

The present invention relates to a method for predicting a disease outbreak. This method includes the steps of collecting data by the remote data interface (120), validating the obtained data by the remote data interface (120), analysing data and computing new parameters by a data analysis interface (140), identifying the association between the case, the outbreak and various indicators, and establishing one-to-one relationships between each case and the index case of the outbreak, and one-to-many relationships between each case and other cases of the outbreak by the remote data interface (120). Thereon, a data prediction interface (150) predicts case parameters that include time of the outbreak and number of people affected by the outbreak, severity of the outbreak, and geographical location of an epicentre of the outbreak.

Preferably, the step of predicting case parameters by the data prediction interface (150) includes the sub-steps of obtaining weather, disease, and nearby construction site information, clustering weather, disease and nearby construction site information, obtaining a regression coefficient table, determining a linear trend of a set of data based on the error values of the data, wherein the set of data refers to the weather, disease and nearby construction site information and predicting the parameters of the cases by the generalised linear model.

Additionally, the data prediction interface (150) predicts the severity of the outbreak by the data prediction interface (150) by obtaining weather, disease, and nearby construction site information and obtaining severity of the outbreak based on a decision tree algorithm.

The step of predicting geographical location of an epicentre of the outbreak by the data prediction interface (150) includes the sub-steps of obtaining hotspot, disease, and weather information, predicting variables of geographical location of the epicentre of the outbreak, classifying the variables of geographical location of the epicentre into a plurality of class labels, wherein the class labels refer to a plurality of cases, and instantiating and comparing the classified variables of geographical location of the epicentre with training dataset to determine whether an outbreak really happens or not.

### BRIEF DESCRIPTION OF THE DRAWINGS

The accompanying drawings, which are incorporated in and constitute a part of the specification, illustrate embodiments of the invention and, together with the description, serve to explain the principles of the invention.

FIG. 1 illustrates a block diagram of a system (100) for predicting disease outbreaks according to an embodiment of the present invention.

FIG. 2 illustrates a flowchart of a method for predicting disease outbreaks according to an embodiment of the present invention.

FIG. 3 illustrates a flowchart of sub-steps for collecting data by the remote data interface (120) of the method of FIG. 2.

FIG. 4 illustrates a flowchart of sub-steps for validating data by the remote data interface (120) of the method of FIG. 2.

FIG. 5 illustrates an example of an epidemic network.

FIG. 6 illustrates a flowchart of sub-steps for identifying associations of data and creating relationships among cases by the remote data interface (120) of the method of FIG. 2.

FIG. 7 illustrates an example of changing the parameters of an outbreak.

FIG. 8 illustrates a flowchart of sub-steps for predicting parameters of the outbreak by the data prediction interface (150) o the method of FIG. 2.

### DESCRIPTION OF THE PREFERRED EMBODIMENT

A preferred embodiment of the present invention will be described herein below with reference to the accompanying drawings. In the following description, well-known functions or constructions are not described in detail since they would obscure the description with unnecessary detail.

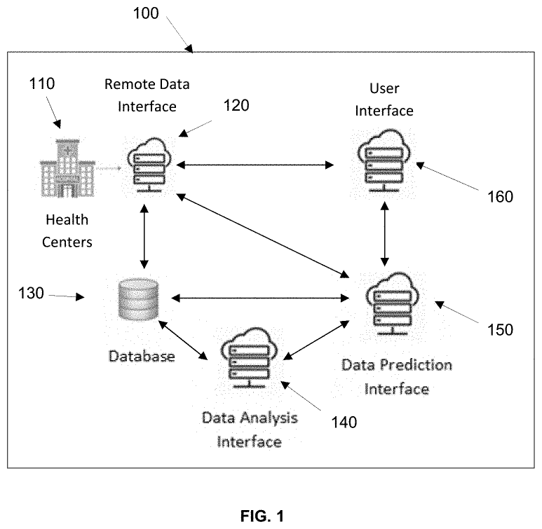

Reference is initially made to FIG. 1 which illustrates a block diagram of a system (100) for predicting the outbreak of a disease. The system (100) comprises a remote data interface (120), a database (130), a data analysis interface (140), a data prediction interface (150), and a user interface (160). The system (100) predicts the outbreak of vector-borne diseases, which are transmitted by vectors such as insects and animals. The system (100) also processes complex information regarding the disease in seconds. Processing through complex information would take health officials weeks to go through. Hence, the system (100) saves valuable time and resources.

The remote data interface (120) is configured to collect clinical data from health centres (110). The health centres (110) may include but not limited to hospitals, clinics, ministries, or any other source of clinical information. The data collected comprises data regarding the patient such as case number, date, notification date, age, gender, hospital entry and exit dates. The data further comprises information regarding the location of the case such as the neighbourhood, postcode, latitude, longitude, area, and clinical information of the case such as results from lab tests or other forms of clinical data. The before mentioned data may be collected at different intervals and time periods. Furthermore, the data collected may be in the form of reports, papers, e-mails, pagers, fax, and messages.

The remote data interface (120) is also configured to obtain other information related to the conditions of the environment such as weather, humidity, rain, temperature, and other environmental variables. Other information regarding the trends of the disease including but not limited to the density of the vector, the movement of vectors bearing the disease, the wind direction, and the trend of cases may also be obtained by the remote data interface (120). The remote data interface (120) is connected to the data analysis interface (140) to send data for analysis. Likewise, the remote data interface is connected with the data prediction interface (150) to send data to predict the disease outbreak.

The remote data interface (120) is further configured to filter the data collected from health centres (110) and other sources, extract parameters relevant to the disease and eliminate irrelevant parameters to enhance the performance by reducing the amount of data processed. The remote data interface (120) is further configured to validate the data by creating an epidemic network and analysing symptoms and neighbouring cases. Preferably, the remote data interface (120) may be configured to provide security features to protect the data and prevent access of unauthorised personnel. The security features may include data protection, user permissions, administrations, and encryption.

The remote data interface (120) is configured to check every case and compare the number of cases in a specific range and timespan with a predetermined number. If the number of cases is greater than the predetermined number, the remote data interface (120) creates a database object and stores it in the database (130).

The database (130) which is connected to the remote data interface (120) is configured to store the data regarding the cases, data regarding the disease, and database objects. The database object comprises a one-to-one relationship with the index case of that outbreak, multiple one-to-many relationships with other cases of the outbreak, a begin date which is the date of the first symptom of the first case, and an end date which is the date of the first symptom of the newest outbreak case. The database object may be in different forms such as charts, tables, clusters, sequences. The database (130) may be in the form of a server, cloud storage, solid-state drives, hard disks, compact disks, or other database configurations. The database (130) may also be a plurality of devices such as multiple servers connected via a communication method. The database (130) may be further configured to operate offline if an online connection is not available.

Furthermore, the database (130) may be a relational database, which is a database with a structure that recognises relationships among stored items. Preferably, this database (130) is connected to the user interface (160) to provide advanced searching capabilities that allow users to search for information in the database (130) via the user interface (160). It is also preferable for the data obtained to be viewed by the user via the user interface (160) in different methods such as plots, charts, tables, and graphs. Geographical locations, epidemic networks, and other data may also be plotted on a map to give a general view of the cases. Additionally, a pivot table may be constructed to allow users to sort and filter the data.

The database (130) is further connected to the data analysis interface (140) which is configured to use the obtained data to compute new parameters such as mean, median, maximum, minimum, standard deviation, and average. The computed parameters are used to predict the outbreak as well as train prediction models used in the data prediction interface (150). The computed parameters are sent to the database (130) for storage, the data prediction interface (150) for predicting the outbreak, and the user interface (160) for visualisation.

The data prediction interface (150) which is connected to the data analysis interface (140) and the remote data interface (120) receives parameters from the data analysis interface (140) and the remote data interface (120) and utilises prediction algorithms to predict parameters of the outbreak of a disease. The prediction algorithms utilise parameters of existing cases as well as other indicators to predict the parameters of an outbreak in advance. The predicted parameters are used to train the models used in the data prediction interface (150).

The data prediction interface (150) is further connected to the user interface (160) which is configured to plot the predicted parameters on a geographical map, scatter plots, bubble plots, simulations, and other types of visualisation. The user interface (160) is also configured to allow the users to edit the details of the cases, discard cases, and manipulate it in different methods. Some implementations of the present invention may include configuring the user interface (160) to send notifications to health centres (110) to request missing information, further data for analysis, or any other information.

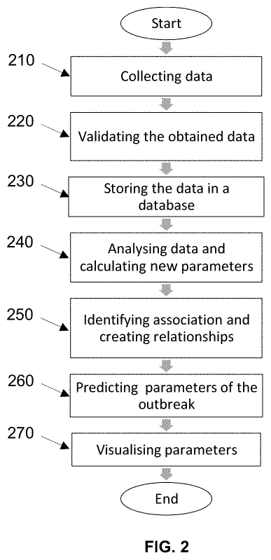

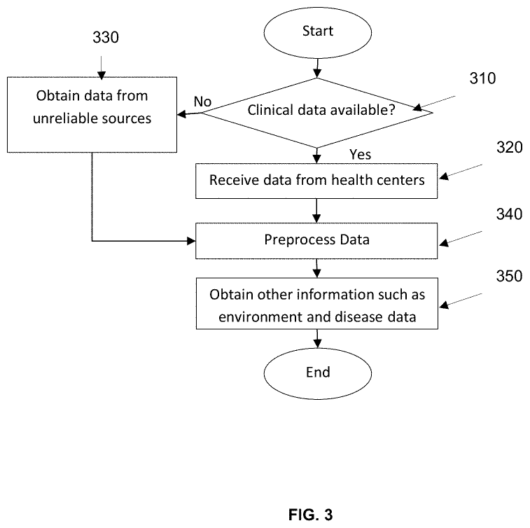

Reference is now made to FIG. 2, wherein FIG. 2 illustrates a flowchart of a method for predicting an outbreak according to an embodiment of the present invention. Initially, data is collected by the remote data interface (120) in step 210. The collected data comprises clinical data that is collected from health centres (110) and other information related to the conditions of the environment such as weather, humidity, rain, temperature, and other environmental variables. The health centres (110) include any source of clinical data such as hospitals, clinics, or ministries of health, the sub-steps of collecting data will be further explained in reference to FIG. 3.

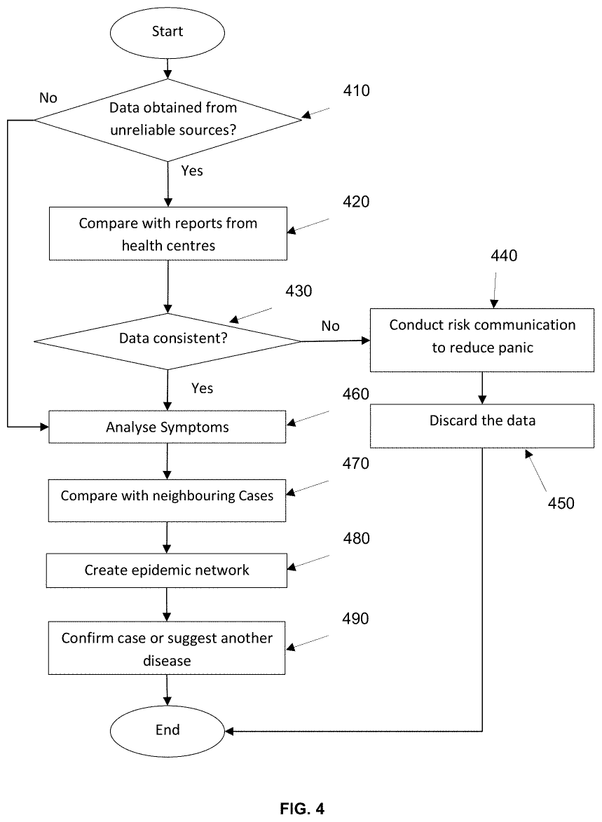

The remote data interface (120) validates each case by analysing the symptoms and comparing the case with neighbouring cases in step 220. The data is validated to confirm the case or suggest another disease for that case based on the neighbouring cases. The sub-steps of validating the clinical data collected from health centres (110) and unreliable sources will be explained in reference to FIG. 4.

Thereon, new parameters are computed by the data analysis interface (140) using the data in the database (130) as in step 240. The computed parameters include but are not limited to the mean, maximum, minimum, average, standard deviation, and any other type of statistical parameters. The computed parameters may aid in creating and training the prediction models used by the data prediction interface (150). Thereon, the computed parameters may be stored in the database (130) or sent to the user interface (160) for visualisation.

Subsequently, the association and relationships between the cases, the outbreak, and various indicators are identified by the remote data interface (120) in step 250. The remote data interface (120) examines every case, compares the parameters of the case with predetermined parameters, and creates relationships with other neighbouring cases. The sub-steps of identifying associations and creating relationships will be further explained in relations to FIG. 4. The identified relationships and associations between cases are also stored in the database (130).

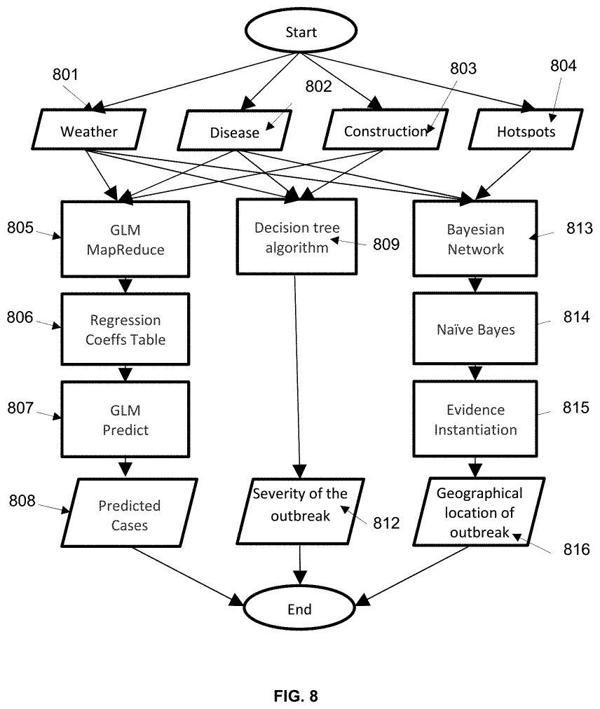

The validated case parameters from step 220, the new parameters computed from step 240, and the relationships created from step 250 are then used to predict parameters of the outbreak by the data prediction interface (150) as in step 260. The predicted parameters may include but not limited to the geographical location of the epicentre of the outbreak, the time of the outbreak, the severity of the outbreak, the number of people affected by the outbreak, the potential spreading area of the outbreak, the probability of the outbreak, and other parameters. The data prediction interface (150) uses multiple algorithms for predicting parameters. The sub-steps of predicting parameters of the outbreak by the data prediction interface (150) will be further explained in relation to FIG. 8.

The predicted parameters may be further evaluated in terms of accuracy, specificity, sensitivity, and other evaluation parameters. The evaluations may be used to train the prediction model or viewed by experts in order to modify parameters of the models used in the data prediction interface (150) to optimise its performance.

Finally, the predicted parameters are sent to the user interface (160) and displayed on a map as a scatter plot, bubble plot, a simulation, or other forms of visualisations in step 270.

Some implementations of the present invention may include the step of alerting users or health offices of the start of a new outbreak by the user interface (160). The alerts may be in the form of a report, pager, fax, sound alert, maps, or any alerting method. The alerts may contain the location, time, details of the disease.

FIG. 3 illustrates a flowchart of sub-steps for collecting data by the remote data interface (120) of step 210 of the method of FIG. 2. Initially, the remote data interface (120) determines whether clinical data is available from health centres (110) as shown in decision 310. If clinical data from health centres (110) is available, the remote data interface (120) imports the data from health centres (110) as in step 320.

The data is then pre-processed by the remote data interface (120) in step 330 by removing irrelevant parameters that are not used in the prediction. Other pre-processing steps include but are not limited to merging two datasets from different sources, eliminating duplicate reports, eliminating reports with missing information, eliminating reports with irrational values, normalizing data obtained from reports. For instance, the latitude and longitude of the case are enough for geographically locating the case. Other information such as the district, sector, province, postcode, and neighbourhood are all redundant. Hence, redundant data requires additional processing time and extra storage space.

Otherwise, if clinical data from health centres (110) is not available as in decision 310, the remote data interface (120) obtains data from other sources as in step 330. The other sources are referred to as unreliable sources due to the fact that other sources may also contain false information and rumours. The unreliable sources include sources such as social media, news, mobile applications and search engines. The data is then pre-processed by the remote data interface (120) as mentioned in step 340.

After pre-processing the data, the remote data interface (120) then collects other information that is used for predicting the outbreak in step 350. The other information includes geocoding information, weather information, landmark information, geographic information, and socioeconomic information. This information may be obtained from various sources such as online sources, organizations, and statistical data from local offices.

FIG. 4 illustrates a flowchart of sub-steps for validating data by the remote data interface (120) of step 220 of the method of FIG. 2. The remote data interface (120) determines whether the data is obtained from unreliable sources in decision 410. If the data was obtained from unreliable sources as in decision 410, the remote data interface (120) determines the validity of the data by comparing the data received from unreliable sources with reports from health centres (110) that are confirmed by lab tests as in step 420.

The remote data interface (120) determines whether the data is consistent or not with reports from health centres in decision 430. If a plurality of reports obtained from unreliable sources is inconsistent with the reports from health centres as in decision 430, the remote data interface (120) alerts the local government in order to conduct risk communication in step 440. The risk communication is conducted to reduce the panic that resulted from false information obtained from sources such as news and social media. The data is then discarded by the remote data interface (120), and the remote data interface (120) proceeds to validate the next set of data.

On the other hand, if the data from unreliable sources is consistent with reports from health centres as in decision 430, the remote data interface (120) proceeds to analyse the symptoms of the case in step 460 and compare the case with neighbouring cases in step 470. After that, an epidemic network is created by the remote data interface (120) in step 480.

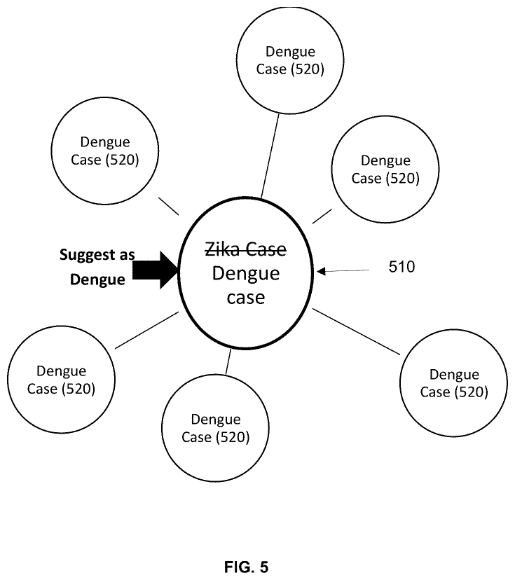

An example of the epidemic network of a new case (510) is illustrated in FIG. 5. Initially, the case was diagnosed as Zika. However, all the nearby cases (520) in that time span were confirmed to be Dengue cases. The remote data interface (120) analyses the symptoms of the new case (510) and links the case with neighbouring cases (520). Therefore, the remote data interface (120) suggests that the case 510 is most likely to be a Dengue case after analysing nearby cases (520).

Referring back to FIG. 4, after analysing the symptoms and nearby cases and creating an epidemic network, the disease may be confirmed if the symptoms and test results of the case are consistent with the symptoms of the disease, and the symptoms of the case are consistent with neighbouring cases in step 490. Another disease may be suggested for the case if the lab test results of that case or neighbouring cases indicate symptoms of another disease.

For example, in the case of dengue reports, the remote data interface (120) examines the results of some lab tests such as immunoglobulins blood tests and non-structural protein 1 test to confirm that the case is dengue. If the results do not indicate a dengue case, the remote data interface (120) suggests another disease such as zika that is consistent with the test results.

If the data is not obtained from unreliable sources as in decision 410, the remote data interface (120) analyses the symptoms of the case in step 460 and compares the case with neighbouring cases in step 470. After analysing the symptoms of the case and comparing the case with neighbouring cases, the remote data interface (120) creates an epidemic network in step 480. The remote data interface (120) then proceeds to confirm the case or suggest another disease in step 490.

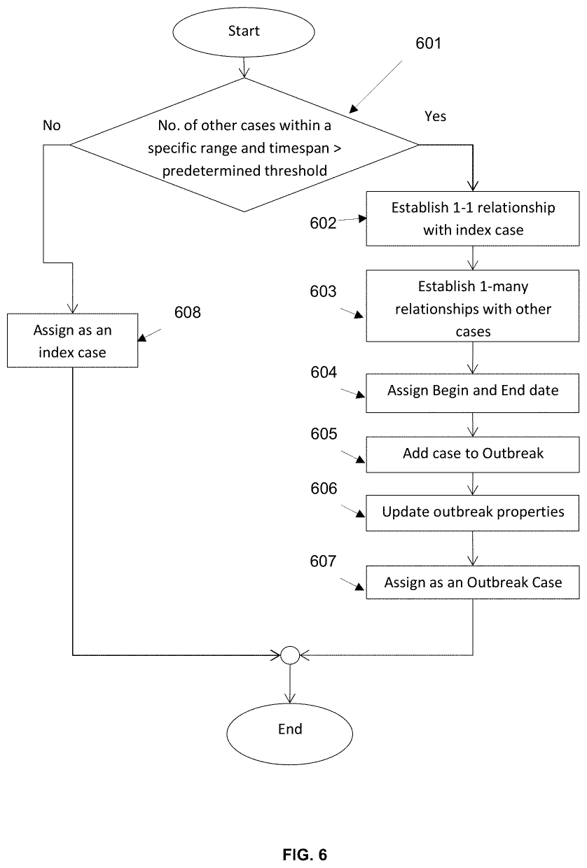

FIG. 6 illustrates a flowchart of sub-steps for identifying the association of data and creating relationships in step 250 of the method of FIG. 2. For each case, the remote data interface (120) compares number of cases in a specific range and timespan with predetermined thresholds of an outbreak as in decision 601. The predetermined thresholds are preferably established by consulting experts or health centres (110).

If the number of cases within a specific range and timespan is more than the predetermined threshold in decision 601, a one-to-one relationship with the index case of that outbreak is established in step 602. The one-to-one relationships are established to assign the case to an outbreak of which the index case is the epicentre of the outbreak. Likewise, multiple one-to-many relationships with other cases of the outbreak are established in step 603. The one-to-many relationships are established to determine the propagation of the outbreak. For instance, if a case is reported within the range of an outbreak case but not within the range of the index case, the outbreak may be considered as spreading or propagating to nearby areas. In this case, the reference is not only set to the index case, but it is also set to other cases that have a one-to-one relationship with the index case.

The relationships may be established in methods that include more complex operations such as convoluted neural networks, relationship matrices, causality assessment algorithms, correlation matrices. An outbreak is stored in the database (130) as a database object and is used to store information about the outbreak including its begin date, end date, and the cases of that outbreak. The outbreak is assigned a begin date which is the date of the first symptom of the first case, and an end date which is the date of the first symptom of the newest outbreak case in step 604. The case is assigned as an index or an outbreak case. An index case is the oldest case in the outbreak, while outbreak cases are subsequent cases in that outbreak.

For every case in the database (130), the remote data interface (120) adds the case to an existing outbreak as in step 605. The remote data interface (120) also updates parameters of an existing outbreak including its index case, number of cases in the outbreak, begin date, and end date as shown in step 606. Subsequently, the case is assigned as an outbreak case in step 607.

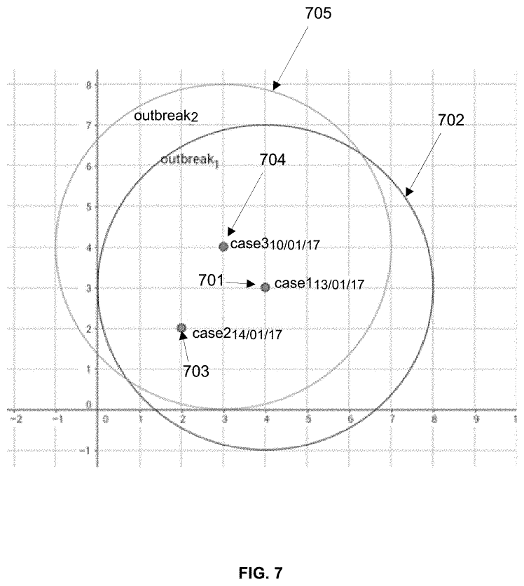

FIG. 7 illustrates an example of updating the parameters of the disease. In this example, the first case (701) was reported and set to be an index case, hence the radius of the outbreak was set in the neighbourhood (702) and the begin date is the date of the first case (701). A second case (703) is reported in the following day and the remote data interface (120) determined that it is within the predetermined radius of the first case, hence the second case is determined to be an outbreak case related to the first case (701) which is the index case. The end date of the outbreak will be the date of the second case (703) which is the case with the latest date.

Later on, another case (704) was reported and was determined within the predetermined radius (702) of the first case (701). However, the time of the occurrence of the third case (704) preceded the time of the first case's occurrence (701), hence it is more likely for the third case (704) to be the index case of the first case (701) because the third case (704) occurred first. Therefore, parameters of the outbreak have to be changed, in this case, the index case of the outbreak is set to be the third case (704) because the third case (704) preceded the first case (701) and the second case (702). In addition, the first case (701) is changed from an index case to an outbreak case. In addition, the begin date is changed to the date of the third case (704), the end date will remain the same, and the radius of the index case is changed to the predetermined range around the third case (704) which is shown as the new neighbourhood (705)

Referring back to FIG. 4, if there are less than a specific number of cases in a specific range and timespan in decision 601, the case is assigned as an index case in step 608. The relationships and associations will be sent to the data prediction interface (150) to predict the parameters of the outbreak as in step 260 of the method of FIG. 2.

FIG. 8 illustrates a flowchart of sub-steps for predicting parameters of the outbreak by the data prediction interface (150) of step 260 of the method of FIG. 2. Initially, the data prediction interface (150) obtains information regarding the weather, disease, nearby construction site and disease hotspot in steps 801, 802, 803, and 804 respectively, wherein the disease hotspots are areas in which the disease is concentrated and spread. The information includes the validated case parameters from the remote data interface (120), new parameters computed by the data analysis interface (140) and relationship created by the remote data interface (120).

The data prediction interface (150) proceeds to utilize the weather information, disease information, and nearby construction site information in a MapReduce model to cluster the information as in step 805. The MapReduce model is an algorithm that is used in big data analytics to cluster big data by splitting the data, processing the data in parallel, and then combining the data. The MapReduce is used as a clustering technique in the present invention.

Subsequently, a regression coefficient table is obtained in step 806 and is used in a generalised linear model for prediction in step 807. A generalised linear model is an algorithm for determining a linear trend of a set of data based on the error values of the data such as root mean square or sum of the square of errors, wherein the set of data refers to the weather, disease and nearby construction site information. Hence, the parameters of predicted cases are obtained by the data prediction interface (150) based on the linear trend in step 808, wherein the parameters of predicted cases include but not limited to the time of the outbreak and the number of people affected by the outbreak.

In addition, the data prediction interface (150) further utilises the weather, disease, and nearby construction site information in a decision tree algorithm for predicting the severity of the outbreak in step 809. The decision tree algorithm is a machine learning algorithm that uses a decision tree to go from observations about an item to come up with a conclusion about a specific value. In relation to the present invention, the observations are the weather, disease, and nearby construction site information of the case and the target variable to be predicted is the severity of the outbreak. The severity of the outbreak is then obtained in step 812.

Moreover, the data prediction interface (150) utilises weather, disease and disease hotspot information in a Bayesian network algorithm to predict variables of the geographical location of the epicentre of the outbreak in step 813. The Bayesian Network is a probabilistic graphical model that is used to predict outcomes given a set of conditions or events. The conditions or events are represented as nodes, wherein the nodes are connected based on their dependency on other nodes. For example, an event that is not connected to any node is independent of the conditions.

The variables of the geographical location of the epicentre of the outbreak are then fed to a Naïve′ Bayes in step 814, wherein Naïve′ Bayes is a Bayesian network model that assigns class labels to problem instances. The class labels are represented as vectors of feature values and are drawn from a finite set. In this instance, the class labels refer to the cases. The Naïve Bayes assumes that the value of each class label is independent of the value of other class labels and classify the variables of the geographical location of the epicentre of the outbreak into the class labels.

The classified variables of the geographical location of the epicentre of the outbreak are used for evidence instantiation in step 815. The classified variables of the geographical location of the epicentre of the outbreak are instantiated and compared with training dataset to determine whether the outbreak really happens or not. The geographical location of the epicentre of the outbreak is then obtained in step 816.

While embodiments of the invention have been illustrated and described, it is not intended that these embodiments illustrate and describe all possible forms of the invention. Rather, the words used in the specifications are words of description rather than limitation and various changes may be made without departing from the scope of the invention.

1. A method for predicting parameters of a disease outbreak is characterised by the steps of:
a) collecting data regarding a disease by a remote data interface, the data includes clinical data from health centres and environmental data from various sources;
b) validating the obtained data by the remote data interface;
c) analysing data and computing new parameters by a data analysis interface;
d) identifying associations and relationships among the disease cases, the outbreak and various indicators by the remote data interface;
e) predicting case parameters by a data prediction interface, wherein the predicted case parameters include time of the outbreak and number of people affected by the outbreak;
f) predicting severity of the outbreak by the data prediction interface; and
g) predicting geographical location of an epicentre of the outbreak by the data prediction interface.

2. The method as claimed in claim 1, wherein collecting data regarding a disease by the remote data interface includes the steps of:
a) determining whether clinical data is available from health centres;
b) importing clinical data from health centres if clinical data from health centres is not available;
c) pre-processing data by removing irrelevant parameters that are not used in the prediction, merging two datasets from different sources, eliminating duplicate reports, eliminating reports with missing information, eliminating reports with irrational values, normalizing data obtained from reports, and other pre-processing steps; and
d) obtaining information used for predicting the parameters of the disease, wherein the information includes geocoding information, weather information, landmark information, and socioeconomic information.

3. The method as claimed in claim 2, wherein if there is no data available from health centres, the remote data interface searches for data from unreliable sources, wherein unreliable sources include news, social media, and other sources.

4. The method as claimed in claim 1, wherein validating data obtained by the remote data interface includes the steps of:
a) determining whether the data is obtained from health centres or unreliable sources;
b) if the data is obtained from unreliable sources, comparing the data with reports obtained from health centres;
c) determining whether the data from unreliable sources is consistent with the reports from health centres;
d) if the data is consistent with clinical data from health centres, analysing symptoms of the case;
e) comparing the case with neighbouring cases;
f) creating epidemic networks; and
g) confirming the disease or suggesting another disease for the case.

5. The method as claimed in claim 4, wherein if the data from unreliable sources is not consistent with reports from health centres, the steps include:
a) alerting the local government to conduct risk communication; and
b) discarding the data.

6. The method as claimed in claim 1, wherein identifying the association and relationships between the case, the outbreak and various indicators by the remote data interface includes the steps of:
a) determining whether the number of cases in a specific range and timespan is higher than a predetermined threshold;
b) if the number of cases in a specific range and timespan is higher than a predetermined threshold, establishing a one-to-one relationship with the index case of the outbreak;
c) establishing one-to-many relationships with other cases in the outbreak;
d) assigning a begin date which is the date of the first symptom of the first case, and an end date which is the date of the first symptom of the newest outbreak case;
e) updating the properties of the outbreak including its index case, number of cases in the outbreak, begin date, and end date; and
f) assigning the case as an outbreak case.

7. The method as claimed in claim 6, wherein if the number of cases in a specific range and timespan is lower than a predetermined threshold, assign the case as an index case.

8. The method as claimed in claim 1, wherein predicting case parameters by the data prediction interface includes the steps of:
a) obtaining weather, disease, and nearby construction site information;
b) clustering weather, disease and nearby construction site information;
c) obtaining a regression coefficient table;
d) determining a linear trend of a set of data based on the error values of the data, wherein the set of data refers to the weather, disease and nearby construction site information; and
e) predicting the parameters of the cases by the generalised linear model.

9. The method as claimed in claim 1, wherein predicting severity of the outbreak by the data prediction interface includes the steps of:
a) obtaining weather, disease, and nearby construction site information; and
b) obtaining severity of the outbreak based on a decision tree algorithm.

10. The method as claimed in claim 1, wherein predicting the geographical location of an epicentre of the outbreak by the data prediction interface includes the steps of:
a) obtaining hotspot, disease, and weather information; and
b) predicting variables of the geographical location of the epicentre of the outbreak;
c) classifying the variables of the geographical location of the epicentre into a plurality of class labels, wherein the class labels refer to a plurality of cases; and
d) instantiating and comparing the classified variables of the geographical location of the epicentre with training dataset to determine whether an outbreak really happens or not.

11. The method as claimed in claim 1, wherein the method further includes the steps of:
a) alerting the user of an outbreak by a user interface; and
b) visualising the data by the user interface.

12. The method as claimed in claim 1, wherein the new parameters computed by the data analysis interface mean, maximum, minimum, and standard deviation.
